(19) United States
(12) Patent Application Publication
(10) Pub. No.: US 2007/0141182 A1
(43) Pub. Date: Jun. 21, 2007

(54) Combination of multiple nonteroidal antiinflammatory drugs and muscle relaxants for local treatment of musculoskeletal pain

(76) Inventor: Sarfaraz K. Niazi, Deerfield, IL (US)

Correspondence Address: SARFARAZ K. NIAZI, 20 RIVERSIDE DRIVE, DEERFIELD, IL, 60015, US

(21) Appl. No.: 11/306,246

(22) Filed: Dec. 20, 2005

(51) Int. Cl. A61K 36/81; A61K 36/31; A61K 31/7076; A61K 31/70; A61K 31/519; A61K 31/538; A61K 31/554; A61K 31/553

(52) U.S. Cl. 424/755; 514/569; 514/570; 514/230.5; 424/760; 514/211.07; 514/159; 514/355; 514/25; 514/262.1; 514/509; 514/47; 514/356

(57) ABSTRACT

This invention relates to pharmaceutical compositions for use in the treatment of pain and inflammation and the treatment of muscle spasms and associated pain, soreness and tightness of muscles in mammalian organism, said composition comprising: (i) an analgesically and anti-inflammatory effective amounts of five NSAIDs and (ii) an amount effective in the treatment of muscle spasms of at least one of the muscle relaxants, wherein (iii) the said preparation is applied locally to muscles and optionally contains absorption and blood flow enhancers.

Nonsteroidal antiinflammatory drugs (NSAIDs) are among the most commonly prescribed therapeutic agents worldwide (Current Opinion in Rheumatology 1991; 3:336-40). NSAIDs rank fifth among the most frequently prescribed medications in the United States. Effective in the treatment of pain, inflammation and fever, NSAIDs are believed to derive many of their properties from a capacity to inhibit prostaglandin synthesis and cyclooxygenase activity (American Pharmacy 1992; 3: 41-47). The currently available NSAIDs, nonspecific cyclooxygenase (COX) inhibitors, deplete tissue protective prostaglandins. As a result, 25% of those taking NSAIDs on a regular basis experience side effects of which 5% are serious: ulcers and gastrointestinal bleeding.

A large number of nonsalicylate NSAID preparations are approved for use in the United States; the continuing search for the ultimate NSAID is driven by the high demand for better therapies for treating musculoskeletal syndromes and by dissatisfaction with the efficacy or the side effects of available NSAIDs. Although certain newer agents may offer safety and tolerability benefits over the older agents, NSAIDs continue to be one of the most common groups of drugs associated with serious adverse effects. Patients can vary in their response to NSAID therapy. If one NSAID seems ineffective after a dose adjustment a change in the choice of NSAID is often made.

NSAIDS can be characterized into five primary groups: (1) the propionic acids; (2) the acetic acids; (3) the fenamic acids; (4) the biphenylcarboxylic acids; and (5) the oxicams.

“Propionic acid NSAIDS” as defined herein are non-narcotic analgesics/nonsteroidal antiinflammatory drugs having a free —CH(CH₃)COOH group, which optionally can be in the form of a pharmaceutically acceptable salt group, e.g., —CH(CH₃)COO⁻Na⁺. The propionic acid side chain is typically attached directly or via a carbonyl function to a ring system, preferably to an aromatic ring system. Exemplary propionic acid NSAIDS include: ibuprofen, indoprofen, ketoprofen, naproxen, benoxaprofen, flurbiprofen, fenoprofen, fenbufen, pirprofen, carpofen, oxaprozin, pranoprofen, miroprofen, tioxaprofen, suprofen, alminoprofen, tiaprofen, fluprofen, and bucloxic acid. Structurally related propionic acid derivatives having similar analgesic and antiinflammatory properties are also intended to be included in this group. As is evident from the structural formula above, profens exist in enantiomeric forms. NSAIDs from other classes may also exhibit optical isomerism. The invention contemplates the use of pure enantiomers and mixtures of enantiomers, including racemic mixtures, although the use of the substantially optically pure enantiomer will generally be preferred.

“Acetic acid NSAIDS” as defined herein are non-narcotic analgesics/nonsteroidal antiinflammatory drugs having a free CH₂COOH group (which optionally can be in the form of a pharmaceutically acceptable salt group, e.g., CH₂COO⁻Na⁺, typically attached directly to a ring system, preferably to an aromatic or heteroaromatic ring system. Exemplary acetic acid NSAIDS include: ketorolac, indomethacin, sulindac, tolmetin, zomepirac, diclofenac, fenclofenac, alclofenac, ibufenac, isoxepac, furofenac, tiopinac, zidometacin, acematacin, fentiazac, clidanac, oxpinac, and fenclozic acid. Structurally related acetic acid derivatives having similar analgesic and antiinflammatory properties are also intended to be encompassed by this group.

“Fenamic acid NSAIDs” are non-narcotic analgesics/nonsteroidal antiinflammatory drugs having a substituted N-phenylanthranilic acid structure. Exemplary fenamic acid derivatives include mefenamic acid, meclofenamic acid, flufenamic acid, niflumic acid, and tolfenamic acid.

“Biphenylcarboxylic acid NSAIDS” are non-narcotic analgesics/nonsteroidal antiinflammatory drugs incorporating the basic structure of a biphenylcarboxylic acid. Exemplary biphenylcarboxylic acid NSAIDs include diflunisal and flufenisal.

“Oxicam NSAIDs” are N-aryl derivatives of 4-hydroxyl-1,2-benzothiazine 1,1-dioxide-3-carboxamide. Exemplary oxicam NSAIDs are piroxicam, sudoxicam and isoxicam.

All of these drugs listed above work primarily by inhibiting the enzyme cyclooxygenase (prostaglandin synthase), which is responsible for converting arachadonic acid to prostaglandins (Current Opinion in Rheumatology 1994; 6:38-251). NSAIDs may also exert their analgesic effects by inhibiting prostaglandin synthesis, because it is believed that pain receptors are prostaglandin sensitive. These agents also have important pharmacologic actions unrelated to their effects on prostaglandins. Among these properties is the ability to inhibit the release of mediators of inflammation from neutrophils and macrophages (Seminars in Arthritis and Rheumatism. 1990; 19 (4, Suppl 2): 1-5). These effects are due to the ability of NSAIDs to intercalate into the lipid bilayer of the plasma membrane and thereby disrupt protein-protein and protein-lipid interactions critical for cell responses (e.g., calcium translocations, membrane phospholipid turnover) (Current Opinion in Rheumatology 1991; 3:336-40). Increasing evidence indicates that NSAIDs may use several alternative mechanisms to achieve an anti-inflammatory/analgesic effect. Inhibition of leukotriene production, blockade of oxygen-free radical formation, and interference with protein-protein interactions are several proposed non-cyclooxygenase-dependent mechanisms (N Eng J Med 1991; 324 (24): 1716-25). The existence of the alternate pathways of action is supported by the efficacy of nonacetylated salicylates (i.e., salsalate and magnesium choline trisalicylate), which only weakly inhibit prostaglandins, yet are effective anti-inflammatories. Note that the most potent (and likewise most toxic) NSAIDs cause the highest degree of prostaglandin antagonism. There are no studies relating the degree of cyclooxygenase inhibition with the anti-inflammatory efficacy in individual patients.

It is this difference in the mechanism of action of NSAIDs among the different classes and even within a given class that prompted this invention. It is postulated that if several NSAIDs are administered concomitantly, a better response is expected since a multitude of mechanisms of action will be initiated by this combination of drugs. However, studies demonstrate that oral administration of combination of NSAIDs exacerbates the toxic effects of NSAIDs, particularly with reference to COX-1 suppression. It was therefore necessary to utilize a drug delivery route that eliminates the COX-1 suppression toxicity. Topical administration of combination of NSAIDs offers this advantage, provided the formulation is capable of delivering the drugs to the site of action and not to the deeper tissue to keep the systemic effect to minimal. The magnitude of the problem and severity of complications in the use of NSAIDs has prompted a search for a safer product. This has led to the development of diclofenac/misoprostol, or “arthrotec”, a drug, which provides the standard anti-inflammatory activity of the NSAID diclofenac (voltaren) with the gastrointestinal protective effects of the prostaglandin E1 analog misoprostol (cytotec). Another strategy to obviate the toxicity of COX-1 NSAIDs is the development of NSAIDs that act as selective cyclooxygenase-2 (COX-2) inhibitors. Typically, NSAIDs inhibit prostaglandin synthesis by inhibiting both cyclooxygenase-1 (COX-1) and COX-2 isoforms. The constitutive isoform, COX-1, is produced in most tissues, including platelets, kidneys, joint synovium, and gastric mucosa. The inducible isoform, COX-2, is found in inflamed tissue, activated monocytes, and macrophages. Piroxicam, indomethacin, and sulindac have been shown to preferentially inhibit COX-1. Flurbiprofen, ibuprofen, and meclofenamate appear to inhibit both COX-1 and COX-2 equally. Nabumetone and etodolac more selectively inhibit COX-2, and their use has been associated with a lower incidence of gastric lesions when compared with more traditional NSAIDs. Celecoxib and rofecoxib are highly selective and potent inhibitors of COX-2. Recently, serious cardiac effects of COX-2 NSAIDs have resulted in withdrawal of drugs from the market.

Topically applied NSAID products are not readily available in North America because most NSAIDs have lost their patents, which make them unprofitable for drug companies to invest in seeking regulatory approvals. There is also some controversy in the literature on the efficacy of topically applied NSAIDs. Nevertheless, topical NSAIDs are widely available and are prescribed extensively in other regions of the world. Topical NSAIDs have four major advantages: 1) higher concentrations of NSAIDs are delivered to the desired site (e.g. up to 100 times higher NSAID occurs in synovial fluid compared to that from NSAID blood levels; 2) only 1-3% of NSAID is systemically absorbed, reducing the possibility of gastrointestinal upset or ulcers; 3) low blood levels reduce the incidence of drug interactions; 4) low skin irritation at the application site. The following list of publications report possible advantages of topical application of NSAIDs:

Gevi, M and Merlo, M., “Ketoprofen lysine by topical route in sports traumatology”, Current Therapeutic Research, 34, 844-850 (1983). Systemic absorption of the active drug after cutaneous application of a 5% gel of ketoprofen lysine was determined in 5 healthy volunteers and was shown to be very low (about 1%). A clinical trial was carried out on 30 sporting patients with various traumatological affections. Spontaneous pain, pain at passive movement, and at pression, swelling were evaluated before and after treatment. The new dosage form was endowed with a high analgesic and anti-inflammatory activity after topical use. Therapy was most effective against spontaneous pain with patients in sports and the very good results obtained on swelling confirmed its high anti-edema activity. The gel was well tolerated with neither topical nor systemic side effects being reported. It was concluded that 5% ketoprofen gel was very useful both as a resolutive and as a supporting therapy of surgery, plaster, or electromedical applications.

Ballerini, R., Casini, A. et al., “Study on the absorption rates of ketoprofen topically—administered in man: Comparison between tissue and plasma levels”, Int. J. Pharm. Res., VI, 69-72 (1986). The transcutaneous passage of ketoprofen after a topical gel administration and its distribution to the inner part of the knee joint was evaluated in 6 patients. The ketoprofen concentration detected was 4.7 mcg/g in the intra-articular adipose tissue, 2.35 mcg/g in the capsular sample and 1.4 mcg/g in the synovial fluid. Plasma samples were also examined. The fluid samples from patients using topical ketoprofen were about 100 times higher in tissues than in plasma drawn at the same time. Since synovial fluid concentrations were also 100 times higher than in plasma, it is assumed that this occurred due to transcutaneous absorption from topical ketoprofen.

Flouvat, B., Roux, A., Dehotal-Landes, B., “Pharmacokinetics of ketoprofen in man after repeated percutaneous administration”, Arzneimittel Forschung, 39, 812-815 (1989). A topical formulation of ketoprofen was tested on 10 healthy subjects receiving a daily dose of 2.5% ketoprofen gel corresponding to 375 mg of the oral dose. Plasma samples were collected after the first dose and after 10 days of chronic treatment. Urine was also collected. Plasma concentrations were only 2.6% of the daily dose applied. No sign of local intolerance was noted.

Rau, R. and Hockel, S., “Piroxicam gel versus diclofenac gel in activated gonarthrosis”, Fortschr. Med., 107, 485-488 (1989). In a single blind comparative study 97 patients suffering from activated gonarthrosis were treated with either 4×40 mg of diclofenac gel or 4×5 mg piroxicam gel. Owing to protocol violations, 28 patients were not included in the statistical evaluation. During the course of the treatment, a marked decrease in signs and symptoms was observed in the 69 patients included in the evaluation. In 80% of the patients, the treating physician assessed the efficacy of piroxicam gel as “good” or “excellent”, in only 20% it was assessed as “moderate”. In the diclofenac group, assessment of the results was positive in 74% of the patients. In 24% of the patients of this group, the physician's assessment of the success of the treatment was “not satisfactory”. The majority of physicians and patients were satisfied with the toleration of the drugs used in this study. Only 2 patients (5.6%) of the piroxicam group and 4 patients (12.2%) of the diclofenac group were critical of local tolerance. The results of this clinical trial show that the preparations used here are appropriate for local therapy of distortions of the ankle joint.

Baixauli, F., Ingles, F., et al., “Percutaneous treatment of acute soft tissue lesions with naproxen gel and ketoprofen gel”, J. Int. Med. Res., 18, 372-378 (1990). A randomized independent group, single-blind study was performed to compare the analgesic efficacy and the local and cosmetic tolerability of 3-5 cm of 10% naproxen gel with 10% ketoprofen gel in 30 patients complaining of moderate or severe pain due to acute soft tissue lesions. Both drugs were administered topically and were applied to the painful area at once every 12 hours as required. Efficacy and tolerability of both naproxen gel and ketoprofen gel were comparable, although naproxen gel produced a significantly greater reduction in pain on deep palpitation by the third day.

White, S., “Topical Non-steroidal Anti-inflammatory Drugs (NSAIDs) in the Treatment of Inflammatory Musculoskelatal Disorders”, Prostaglandins, Leukotrienes and Essential Fatty Acids, 43, 209-22 (1991). The study showed that benzydamine HCl 3% cream clinically improved reduction of painful and inflammatory symptoms of traumatic injuries of soft tissues and joints. Piroxicam gel, 0.5%, also yielded clinical improvement in patients with traumatic injuries and osteoarthritis of the knee and muscular pain. Felbinac gel, 3%, alleviated pain in patients with soft tissue injuries. Adjunct therapy using ultrasound was useful. Pharmacokinetic data showed that the agents penetrate the skin and reach the underlying tissues, including synovial fluid. Plasma levels after topical administration are low. It is concluded that topical NSAIDs are particularly useful for the short-term treatment of acute musculoskeletal pain and inflammation and have less, and less serious, side effects than oral NSAIDs.

Willimann, H., Walde, P., et al., “Lecithin organogel as matrix for transdermal transport of drugs”, J. Pharm. Sci., 81, 871-874 (1992). Organogels obtained by adding small amounts of water to a solution of lecithin in organic solvents were studied as matrices for the transdermal transport of drugs. Gels obtained from isopropyl palmitate and cyclohexane was used (molar ratios of water to lecithin of 3 and 12, respectively). Preliminary histological studies showed that the gels had no harmful effects when applied to the skin for prolonged periods. Data relative to the stability of the organogels with time were also presented. Scopolamine and broxaterol were used as model drugs and the transdermal experiments were done with a Franz diffusion cell system using human skin obtained from plastic surgery. The transport rate of scopolamine obtained with the lecithin gels was about one order of magnitude higher than that obtained with an aqueous solution of the drug at the same conc. In contrast, the transport rates of scopolamine obtained with the micro emulsion solution prior to gelation (molar ratio of water to lecithin, were not different from those obtained with the gel. The same variations in transport rates were observed for broxaterol in which case the flux through the skin was directly proportional to the conc. of drug in the gel. At a conc. of broxaterol of 75 mg/ml in the donor gel, the flux was 47 μg.h⁻¹ cm⁻². Because preliminary results showed that transdermal transport is successful with amino acids and peptides also, it was concluded that lecithin gels maybe efficient vehicles for the transdermal transport of various drugs.

Airaksinen, L., Venalainen, et al., “Ketoprofen 2.5% gel versus placebo gel in the treatment of acute soft tissue injuries”, T. J. Clin. Pharm. Therap. & Toxicol., 31, 561-563 (1993). A parallel double-blind placebo-controlled and randomized clinical study in a single center was done with 2.5% ketoprofen gel in treating soft tissue injuries. Patients applied the gel twice daily for 7 days. Assessments were made on the 3rd and 7th days. The study group consisted of 29 patients and 27 patients in the control group. Pain at rest was significantly relieved in the ketoprofen group whereas the difference was insignificant in the placebo group. No difference in side effects was noticed between the groups. Thus, ketoprofen gel appears to be safe and superior to placebo in the treatment of soft tissue injuries.

Peacock, M., Rapier, C., “The topical NSAID felbinac is a cost effective alternative to oral NSAIDs for the treatment of rheumatic conditions”, Brit. J. Med. Econ., 6, 135-142 (1993). A cost-effectiveness analysis was conducted to compare the basic drug cost, the shadow cost (treatment of peptic ulcers) and the total cost of treating 1000 patients with a month's supply of either an oral NSAID, the topical NSAID, Traxam (Lederle Labs, UK), or the combination product Arthrotec (diclofenac/misoprostol, Searle). A published treatment decision model was used to assess the costs associated with developing an ulcer due to NSAID treatment. The model showed that the shadow cost of treating the peptic ulcers resulting from administration of oral NSAIDs far outweighed the basic cost of these drugs. The large difference in the total treatment costs of oral NSAIDs and Traxam suggests that significant cost savings for both hospital and GP budgets could be made if the topical NSAID, Traxam was prescribed instead of an oral preparation.

Singh, P. and Roberts, M. S., “Skin Permeability and Local Concentrations of Nonsteroidal Anti-inflammatory Drugs after Topical Application”, J. Pharmacol. Exptl. Therap., 268, 144-151 (1994). The human epidermal permeabilities of different NSAIDs (salicylic acid, diethyamine salicylate, indomethacin, naproxen, diclofenac and piroxicam) from aqueous solutions was found to be dependent on the drugs lipophilicity. The extent of local delivery of NSAID was assessed by comparing the tissue concentrations obtained below a treated site to those in contralateral tissues. The estimated tissue concentrations after epidermal applications of NSAIDs could be related to their maximal fluxes across epidermis from an applied vehicle.

Hosie, G. and Bird, H., “The Topical NSAID Felbinac versus Oral NSAIDs: A Critical Review”, Europ. J. Rheumatol. Inflamm., 14, 21-28 (1994). Four separate multicentre, double-blind, double-dummy clinical trials have shown that the efficacy of the topical NSAID, felbinac, is equivalent to that of the oral NSAID, ibuprofen, in the treatment of soft tissue injuries, and to that of oral ibuprofen or fenbufen in mild to moderate osteoarthritis. Because of the gastrointestinal problems associated with oral NSAIDs the cost of treatment with topical felbinac is more economical for reasons of efficacy and safety.

Halpern, S. M., “Topical non-steroidal anti-inflammatory drugs: a review if their use and toxicity”, J. Dermatol. Treat., 5, 103-107 (1994). NSAIDs are generally well tolerated and have proved to be efficacious in arthritis and soft tissue injuries with a much-reduced risk of adverse effects compared to their systemic counterparts. Established side effects include local irritation with erythema or dermatitis, and urticarial reactions can occur with all preparations. Photocontact dermatitis is also well recognized especially with ketoprofen and benzydamine. Systemic reactions, particularly gastrointestinal disturbance and asthma, occur occasionally.

Campbell, J. and Dunn, T., “Evaluation of topical ibuprofen cream in the treatment of acute ankle sprains”, J. Accid. Emerg. Med., 11, 178-182 (1994). One hundred patients who presented to the accident and emergency department with an acute ankle sprain were entered into a study to determine the efficacy of topical ibuprofen cream by using a double-blind placebo controlled design in a single type of soft-tissue injury. The subjects were given either topical ibuprofen cream or a placebo cream in addition to the standard management of the department. Patients kept diaries recording walking ability and pain visual analogue scales for resting, standing and walking. A total of 51 patients returned diaries that were suitable for analysis. Patients using the topical ibuprofen cream had significant reduction in pain scores over the first 48 h of treatment.

Evans, J. M. M. and MacDonald, T. M., “Tolerability of Topical NSAIDs in the Elderly”, Drugs and Aging, 9, 101-108 (1995). The purpose of topical NSAIDs is to achieve a high local concentration of the active ingredient at the affected site, with as low a plasma concentration as possible to minimize possible systemic adverse effects. The elderly seem to be more sensitive to the adverse effects of NSAIDs than younger individuals, and this may also be true for topical NSAIDs.

Evans, J. M. M., McMahon, A. D., McGilchrist, M. M., White, G., Murray, F. E. McDevitt, D. G. and MacDonald, T. M., “Topical non-steroidal anti-inflammatory drugs and admission to hospital for upper gastrointestinal bleeding and perforation: a record linkage case-control study”, Brit. Med. J., 311, 22-26, (1995). The population studied was 319,465 people over a 6-year period. Conclusion: In this study topical non-steroidal anti-inflammatory drugs were not significantly associated with upper gastrointestinal bleeding and perforation after adjustment for the confounding effects of concomitant use of oral anti-inflammatories and ulcer healing drugs.

Heyneman, C. A., “Topical Nonsteroidal Antiinflammatory Drugs for Acute Soft Tissue Injuries”, Ann. Pharmacotherap., 29, 780-782 (1995). Patient ratings of topical administration of commercial forms of felbinac, ibuprofen, piroxicam, ketoprofen and indomethacin indicated a superior efficacy of topical NSAIDs over placebo. Topical indomethacin is as effective as therapeutic doses of oral indomethacin.

Grahame, R., “Transdermal non-steroidal anti-inflammatory agents”, Brit. J. Clin. Pract., 49, 33-35 (1995). There is a growing body of evidence attesting to the efficacy of topical NSAIDs in the treatment of acute soft-tissue injury and chronic soft-tissue overuse lesions. In one study of the latter, their use cut the need for local hydrocortisone injection by 50%. Topical NSAIDs may, therefore, be seen as an effective alternative to local steroid injection in cases of soft-tissue rheumatism, either where the injection is not acceptable to the patient or where the doctor has not acquired the necessary techniques. The safety profile of topical NSAIDs is good. Skin reactions are rare, and this apparently applies to all topical NSAIDs currently available. It is logical to treat a local pathological lesion with a local therapy, provided the agent is delivered effectively and sagely to the target organ or tissue. Where this can obviate the risk of life-threatening complications such as gastric hemorrhage, the case for substituting local for systemic medication is overwhelming.

Dreher, F., Walde, P., Walther, P., Wehrli, E., “Interaction of a lecithin microemulsion gel with human stratum corneum and its effect on transdermal transport”, J. Control. Rel., 45, 131-140 (1997). A soybean lecithin microemulsion of isopropyl palmitate and a small amount of water was studied as a matrix for transdermal drug delivery. The percutaneous absorption of indomethacin and diclofenac dissolved in the gel system resulted in steady-state fluxes of about 1 μg h⁻¹ cm⁻². Interaction studies were performed using Fourier transform infrared spectroscopy (FTIR), differential scanning calorimetry (DSC) and low-temperature scanning electron microscopy. These studies indicated that the lecithin gel, particularly in isopropyl palmitate, affects the stratum corneum lipid organization even after 1-day incubation. In vivo human skin irritation tests showed no significant irritantcy.

Vaile, J. H., Davis, P., “Topical NSAIDs for Musculoskeletal Conditions. A Review of the Literature”, Drugs, 56, 783-799 (1998), In recent years, an influx of topical NSAIDs has appeared due to a high incidence of serious gastrointestinal adverse events associated with systemic NSAIDs and the premise that minimization of plasma concentrations of active drug may result in fewer systemic adverse effects. Evidence in humans and animals with topical NSAIDs demonstrates lower plasma concentrations than with systemically administered drugs, while those in soft tissues are still of a magnitude considered consistent with exerting an anti-inflammatory effect. In joints, however, the evidence is less strong, and there is still dispute whether in this case the drug reaches the joint predominantly via the transcutaneous or systemic route. Studies of soft tissue conditions have demonstrated superiority of topical NSAIDs over placebo and suggest equivalent efficacy in comparison with oral NSAIDs. The results are less convincing in the treatment of arthritic conditions and more clinical trials are required. The adverse event profile of topical agents is reasonable: minor cutaneous effects occur in up to 2% of patients but tend to be self-limiting. GI events appear to be infrequent and minor, although long-term studies are required. The initial costs of topical agents tend to be higher than those of oral agents but a cost-effectiveness analysis suggests an overall benefit.

Bareggi, S. R., Pirola, R., De Benedittis, G., “Skin and plasma levels of acetylsalicylic acid: a comparison between topical aspirin/diethyl ether mixture and oral aspirin in acute herpes zoster and postherpetic neuralgia”, Eur. J. Clin. Pharmacol., 54, 231-235 (1998). Skin and plasma levels of ASA and SA after topically administered ASA/diethyl ether mixture (ADE) in acute herpetic neuralgia and postherpetic neuralgia. Oral ASA (500 mg) or topical ADE (750 mg) was administered to 19 patients and the analgesic effect was assessed by means of a visual analogue scale (VAS). After ADE application, the analgesic effect was very rapid and VAS scores were lower than at baseline. Pain significantly decreased by 82.6% after topical and 15.4% after oral ASA. After ADE, 95% of the patients had excellent or good pain relief, but after oral administration 79% had a poor response. Skin concentrations of ASA, but not SA, after ADE were about 80- to 100-fold those after oral administration. Levels of ASA and SA in plasma after oral administration were similar to those previously found, but after ADE were undetectable or very low. Patients with excellent pain relief showed a trend towards higher ASA skin concentrations.

Grace, D., Rogers, J., Skeith, K., Anderson, K., “Topical diclofenac versus placebo: a double blind, randomized clinical trial in patients with osteoarthritis of the knee”, J. Rheum., 26, 2659-2663 (1999). Seventy patients completed a double blind, randomized, placebo-controlled, parallel group design 2 week clinical trial to determine the efficacy of topical 2% diclofenac in lecithin organogel (PHLOJEL) in the treatment of pain associated with mild to moderate osteoarthritris (OA) if the knee. Patient responses to disease-specific (WOMAC VA3.0) total score and aggregated subscale scores revealed significant improvement (p<0.5) on the aggregated total score and the pain, stiffness, and physical function subscales from baseline to post-treatment for the active treatment group compared to the placebo group. Analysis of gain scores also revealed significant improvement with active versus placebo treatments. Other efficacy measures exhibited no significant differences between or within treatment groups. Thus, a topical formulation of 2% diclofenac in a lecithin organogel appears to have therapeutic value in patients with mild to moderate OA of the knee as determined by responses from the WOMAC osteoarthritis health status measure.

Rosenstein, E. D., “Topical agents in the treatment of rheumatic disorders”, Rheum. Dis. Clin. North Am., 25, 899-918 (1999). Topical drug delivery may be the optimal route for the treatment of localized musculoskeletal disorders because higher drug concentrations can be achieved at the sites of clinical significance. The rationale for the use of topical NSAIDs and capsaicin in the treatment of soft-tissue rheumatic complaints and osteoarthritis of selected joints is reviewed. Although there are extensive, uncontrolled experiences with DMSO that suggests its effectiveness in the treatment of musculoskeletal disorders, controlled trials yield conflicting results. The basis for the use of physical modalities such as phohophoresis and iontophoresis to improve topical drug delivery is summarized.

Several US Patents have been issued regarding topical application of NSAIDs. Examples include: U.S. Pat. No. 6,045,827 to Russell, “Treatment of equine laminitis,” is for compositions and methods of the topical treatment of equine laminitis are disclosed. In particular, combinations of a fast acting nitric oxide (NO) donor, a sustained acting NO donor and an NSAID mixed in a lipid-based carrier are described. The application of such combinations to the affected areas, e.g., the hoofs and surrounding tissues, of an equine afflicted with laminitis provides relief from the debilitating effects of this painful, often life-threatening condition. U.S. Pat. No. Re. 36,419 to Cavanaugh, Jr. “Use of ketorolac for treatment of squamous cell carcinomas of the oral cavity or oropharynx,” provides novel methods for prevention or treatment of primary and recurring squamous cell carcinoma of the oral cavity or oropharynx comprising topical administration, to the oral cavity or oropharynx, of an effective amount of an NSAID, especially a composition administering from about 0.001% to about 0.2% ketorolac to the oral cavity, alone or as an adjunct to surgery and/or radiation therapy. U.S. Pat. No. 5,976,566 to Samour, et al., “Non-steroidal antiinflammtory drug formulations for topical application to the skin,” is for topical alcoholic or aqueous alcoholic gels containing ibuprofen or other NSAIDs, such as, naproxen, in substantially neutral salt form, have enhanced penetration through skin and may provide rapid pain/inflammation relief by including in the formulation 2-n-nonyl-1,3-dioxolane or other hydrocarbyl derivative of 1,3-dioxolane or 1,3-dioxane or acetal, as skin penetration enhancing compound. The amount of propylene glycol may be varied to adjust the initial flux of the NSAID through the skin, especially for ibuprofen, naproxen, and ketorolac. U.S. Pat. No. 5,891,472 to Russell, “Treatment of equine laminitis,” is for compositions and methods of the topical treatment of equine laminitis are disclosed. In particular, combinations of a fast acting nitric oxide (NO) donor, a sustained acting NO donor and an NSAID mixed in a lipid-based carrier are described. The application of such combinations to the affected areas, e.g., the hoofs and surrounding tissues, of an equine afflicted with laminitis provides relief from the debilitating effects of this painful, often life-threatening condition. U.S. Pat. No. 5,888,523 to Galask, et al., “Topical non-steroidal anti-inflammatory drug composition,” is for a means and method for treating inflamed mucosal tissues topically with a non-steroidal anti-inflammatory drug (NSAID). The NSAID is incorporated into a substantive film forming aqueous based carrier system consisting of a water dispersible natural cellulosic polymer and an organic acid. The NSAID composition may further include a water soluble or water hydratable viscosity enhancing agent and/or a plasticizer. The NSAID topical composition is especially effective in treating pain associated with vulvodynia or vulvar vestibulitis. U.S. Pat. No. 5,824,658 to Falk, et al., “Topical composition containing hyaluronic acid and NSAIDS,” is for a method of treating pain topically, said method comprising administering topically to the skin or exposed tissue of a human, a dosage amount of a pharmaceutical composition, said dosage amount comprising (1) a non-steroidal anti-inflammatory drug (NSAID) in a therapeutically effective amount to treat pain of the skin or exposed tissue and (2) a form of hyaluronic acid selected from the group consisting of hyaluronic acid, its non-toxic salts and combination thereof being between 1% and 3% by weight of the composition, characterized in that said dosage amount of said composition is in a dosage form suitable for topical application to the skin or exposed tissue and in a dosage amount in which component (2) exceeds 10 mg/cm.sup.2 of the skin or exposed tissue to which the dosage amount is to be applied, and is in such form that component (2) is immediately available to transport component (1) percutaneously into the epidermis of the skin or exposed tissue to the site of trauma or pathology of pain to be treated, in the skin or exposed tissue, and wherein the molecular weight of the form of hyaluronic acid is less than 750,000 daltons. U.S. Pat. No. 5,807,541 to Aberg, et al., “NSAID/fluoride periodontal compositions and methods,” is for a method for preventing dental caries by administering fluoride and, at the same time controlling periodontal bone loss precipitated by the fluoride, by providing a combination of fluoride and NSAID. Topical medicament compositions including NSAIDS and fluoride are also disclosed. U.S. Pat. No. 5,639,738 to Falk, et al., “Treatment of basal cell carcinoma and actinic keratosis employing hyaluronic acid and NSAIDs,” is for a method of treating a mammal for a condition of the skin or exposed tissue selected from the group consisting of basal cell carcinoma and actinic keratosis. The method consists essentially of topically administering to the site of the condition, more than once per day over a period of days sufficient to treat the condition, a non-toxic effective dosage amount of a composition consisting essentially of (a) a non-steroidal anti-inflammatory drug (NSAID) in an amount sufficient to block prostaglandin synthesis, (b) hyaluronic acid or a pharmaceutically acceptable salt thereof in an amount effective to transport said NSAID into the skin or exposed tissue at the site of the condition. The concentration of the hyaluronic add or salt thereof is between 1-3% by weight of the composition. The molecular weight of the hyaluronic acid or salt thereof is between 150,000 and 750,000 Daltons. A pharmaceutical excipient suitable for topical application is included. The NSAID in the composition may be diclofenac sodium. U.S. Pat. No. 5,626,838 to Cavanaugh, Jr., “Use of ketorolac for treatment of squamous cell carcinomas of the oral cavity or oropharynx,” provides novel methods for prevention or treatment of primary and recurring squamous cell carcinoma of the oral cavity or oropharynx comprising topical administration, to the oral cavity or oropharynx, of an effective amount of an NSAID, especially a composition administering from about 0.001% to about 0.2% ketorolac to the oral cavity, alone or as an adjunct to surgery and/or radiation therapy. U.S. Pat. No. 4,748,174 to Veronesi, “Water soluble salts of an NSAID with meglumine/glucamine,” is for the water soluble acid addition salts of an NSAID, such as acetylsalicylic acid, fenbufen, diflunisal, piroxicam, naproxen, or the like, with either glucamine or meglumine (N-methylglucamine) are useful anti-inflammatory and analgesic drugs, well adopted for parenteral, oral, rectal or topical administration.

The main issues surrounding the development of topical NSAIDs are the sensitization of tissue besides the demonstration of adequate efficacy. For patients without contraindications to oral NSAIDs, topical preparations must be shown to be better than placebo and comparisons to oral NSAIDs typically used for the arthritis under evaluation are required for the comprehensive assessment of efficacy as dictated by the regulatory authorities. For patients unable to take oral NSAIDs, topical preparations must demonstrate efficacy clearly superior to placebo effects. Examples of patients at high risk of oral NSAIDs include: age>65; recent GI bleed; upper GI ulcer, active or recent renal insufficiency; congestive heart failure; hepatic insufficiency. A successful topical formulation will also act locally and not systemically if the benefits of therapy are to be realized. Localized osteoarthritis and regional pain syndromes appear suitable for the use of topical NSAIDs, especially in high-risk patients. A reasonable sensitization level to the topical drug may be acceptable if the drug is demonstrated to be effective and oral NSAIDs are unlikely to be used. For a localized condition, the drug will be applied to a limited surface area. Localized OA of the knees or other large joints should be evaluated separately from nodal osteoarthritis of the hands (Heberdens and Bouchards nodes) or feet (bunions and OA of interphalangeal joints of the toes). Osteoarthritis of large joints such as the hip or spine should be a separate category as it is uncertain whether topical drugs will penetrate into deeper structures. An effect on “deep” joints could raise suspicions that the topical drug is being systemically absorbed. Painful shoulders should be evaluated according to cause, for example glenohumeral osteoarthritis, versus shoulder capsulitis, versus supraspinatus tendinitis versus acromioclavicular osteoarthritis, versus bicipital tendinitis, etc. Similar diagnostic specificity should apply to other local painful conditions, for example epicondylitis, cervical “myositis,” etc. Outcome measures must be clearly defined; for example, pain relief after each application versus the status of the disease after a period of time.

One of the most effective means of treating musculoskeletal pain is to administer in combination, an analgesic and a smooth muscle relaxant. There are two types of relevant muscle relaxants, the polysynaptic depressant type or the non-polysynaptic depressant type. The polysynaptic depressant type of muscle relaxant exerts a selective action on the polysynaptic neuronal systems that control muscle tone, probably blocking or retarding the transmission of nervous impulses in internuncial pathways with the spinal cord and at higher levels. The polysynaptic depressant type of muscle relaxant or compounds with muscle relaxant activity include but are not limited to: carisoprodol, chlorzoxazone, cyclobenzaprine, dantrolene, diazepam, metaxalone, baclofen, quinine, orphenadrine and methocarbanol. The non-polysynaptic depressant type of muscle relaxant includes compounds that act as depressants of muscle-spindle activity and compounds that act on α-motoneurons. The preferred muscle relaxant is cyclobenzaprine hydrochloride or dantrolene sodium or their salts thereof.

In this invention, we have studied the multiplicity of complications in the use of NSAIDs, particularly the enhancement of toxicity when a combination therapy with NSAIDs is tried for oral administration, the fact that the mechanism of action of each class of NSAID may be different and the clinical observations that patients respond differently to different NSAIDs. In designing our invention, we were cognizant of the growing applications of NSAIDs given topically for disorders including basal cell carcinoma, the precancerous, often recurrent, actinic keratoses lesions, fungal lesions, “liver” spots and like lesions (found for the most part in the epidermis), squamous cell tumors, including carcinoma of the oral cavity, metastatic cancer of the breast to the skin, primary and metastatic melanoma in the skin, malignancies and/or tumours in the skin, genital warts (condyloma acuminata), cervical cancer, and HPV (Human Papilloma Virus) including HPV of the cervix, psoriasis (both plaque-type psoriasis and nail bed psoriasis), corns on the feet and hair loss on the head of pregnant women). In all of these situations, a topical formulation of NSAIDs is postulated here.

We therefore formulated a combination of NSAIDs for topical use with distinct advantage of providing adequate drug concentration only in the local synovial fluid, minimal systemic absorption and exposure of the affected body part to broad range of drug molecules acting by different mechanisms.

According to one aspect of the invention described herein abates pain that the patient is experiencing at the paccinian nerve bundles (superficial nerve bundles) at the site of the trauma and/or pathology on/in the exposed tissue and/or skin, the method comprising administering (rubbing on) an effective dosage amount of the composition to the skin and/or exposed tissue.

According to another aspect of the invention there is provided a combination of NSAIDs, preferably one from each major group of NSAIDs, and even more than one from the same category, formulated in a pharmaceutical formulation suitable for direct application to skin or other mucosal surfaces to treat a disease or condition in humans to inhibit prostaglandin synthesis. Even though broad mechanism for the action of NSAIDs is defined, the molecular level interaction that leads to prostaglandin inhibition remains obscure and it is postulated that different classes of NSAIDs induce this effect by a different mechanism resulting in different patient response. The differences in the patient response could also be idiosyncratic wherein each patient has different level of sensitivity due to differences in cellular biochemistry. When molecules of different chemical identity are provided simultaneously at cellular and subcellular level in the tissue, it is postulated that the most optimal effects are obtained since regardless of the idiosyncracy of patient, one or other molecule will form the “best fit.” This rationale is similar to the use of broad range antibiotics when the specific cause of infecting organism cannot be predetermined at the peril of patient. However, this approach is also not the “shot gun” therapy since all molecules are expected, more or less, to yield a similar outcome. While genome mapping offers to us many opportunities to identify, which drug molecule may be the most suitable for a patient among the choice of several molecules, this technology and its applications are decades away. A significant advantage of multiple NSAID therapy given topically, over oral administration, is that the oral administration provides an additive effect on COX-1 inhibition exacerbating the ulceration of gastrointestinal mucosa. Given topically, the side effects of NSAIDs in promoting COX-1 destruction is eliminated. Therefore, the earlier generation NSAIDs become just as safe as the newer generation of NSAIDs, which are several times more expensive such as specific COX-2 inhibitors and recently implicated in serious cardiac toxicity. It is irrelevant therefore in applying the combination of NSAIDs topically to consider their differentiation based on COX-1 and COX-2 activity. Lack of any substantial systemic absorption leads to enhancement of safety allowing prolonged administration of drug. Therefore, we conclude that a combination of NSAIDs from all different classes and including the newer category of COX-2 specific inhibitors will substantially improve the effectiveness of therapy.

According to another aspect of the invention, we have designed formulations, which are systemic independent (there is a lack of a substantial blood level of the drug for example NSAID), but show sufficient penetration into the skin to the site of the trauma and/or pathology. The compositions subsequently clear likely through the lymphatics and are thus available for the treatment of disease and conditions of the lymphatics. Thus, according to another aspect of the invention, we have provided topically applicable percutaneous (intracutaneous) penetrating (best targeting the epidermis) systemic independent acting (not acting essentially through the blood) pharmaceutical compositions.

According to another aspect of the invention, it contains agents that assist in normalizing the absorption profile of various NSAID molecules across skin yet maintaining their superficial activity. Deeper penetration might lead to systemic effect and thus work against the theory on the basis of which this invention is expounded. It has been reported that upon topical administration, NSAIDs show different rates of penetration across the skin (Cordero J A, Camacho M, Obach R, Domenech J, and Vila L. Eur J Pharm Biopharm 2001 March; 51(2):135-142. The In vitro based index of topical anti-inflammatory activity to compare a series of NSAIDs). To normalize the penetrability of various molecules across the skin we have included in our formulation well established promoters of drug absorption from skin including dimethylsufoxide or hyaluronic acid and/or their salts thereof and/or their homologues, analogues, derivatives, complexes, esters, fragments, and sub-units, in a form suitable for administration to the skin and/or exposed tissue in humans. Alternately, the composition may include a substantially water-insoluble transdermal penetration enhancing compound selected from the group consisting of C4 to C16 aliphatic group substituted acetals, hemi-acetals and morpholines and further comprising a physiologically acceptable water soluble polar compound selected from the group consisting of alcohols, glycols, lactams, urea, cycloethylene urea, 1,3-dioxolone, 2-methyl-1-3-dioxolone, 1,3-dioxane, 2 methyl-1,3-dioxane, morpholine, N-methylmorpholine, N-dimethylformamide, dimethylsulfoxide, methylacetate, ethyllactate, monosaccharides, polysaccharides, amino acids, amino alcohols, diethylamine and cycloethylene carbonate. The polar compound may be selected from a group consisting of alcohol, glycol, dioxolane, formamide, carbonate, glucose, urea and mixtures thereof. Alternatively, the polar compound may be an alcohol glycol mixture or lactim. Other compounds include 1-dodecylazacycloheptan-2-one hexamethylenelauramide, N-methyl-2-pyrrolidone, a sucrose aliphatic acid ester, and nonionic surfactants, in an amount of 0.5-10% by weight of the preparation.

According to another aspect of the invention, it contains chemicals, which are known to increase the blood flow to the site of application. It is well-known that vasodilators, rubefacients and other pharmacologically stimulated mechanisms of bringing more flow to the site of application increases absorption of drug across skin since the transport of drug is based on a flux mechanism that is often concentration gradient dependent. Following are examples of preferred embodiments of the composition, without limitation of the choice of ingredients as claimed below. The base for formulating these products can be any type of clear gel or cream or ointment. The blood flow enhancer compound may be selected from the following category but not limited to capsaicin, capsicum extract; erucic acid; nicotinic acid salts; nicotinic acid esters; and nicotinyl alcohols; mustard oil; menthol; methyl salicylate and other compound which are known to cause enhancement of blood flow either by acting as rubefacient or by other pharmacological mechanisms such as vasodilatation or other localized or central pharmacological mechanisms to enhance blood flow to tissue. A major class of vasodilators is those, which act directly in the smooth muscle membrane. They include hydrallazine, verapamil, diltiazem, felodipine, minoxidil, amlodipine, glyceryl trinitrate, isosorbide mononitrate, nicorandil, dipyridamole, multiple actives, alprostadil, oxpentifylline, hydroxyethyl rutosides and tartrazine, adenosine and nimodipine.

According to another aspect of the invention, the invention combines the formulations with compounds that are known to relax smooth muscles. It is known that localized muscle spasms can significantly irritate the nerves which in turn produce muscular tension resulting in a cyclical phenomenon responsible for long treatment schedules for musculoskeletal pain. Muscle spasm of local origin needs to be clinically differentiated from spasticity and sustained muscle contraction in the setting of the central nervous system (CNS) and upper motor neuron injury. Baclofen (Lioresal®) and dantrolene sodium (Dantrium®) are two agents whose use is indicated in the setting of spasticity of CNS etiology. Dantrolene sodium is of particular interest, as its mechanism of action is purely at the muscular level where it serves to inhibit the release of calcium form the sarcoplasmic reticulum. Baclofen is a derivative of gamma-aminobutryic acid (GABA) and is believed to inhibit mono and polysynaptic reflexes at the spinal level. The choice of muscle relaxants includes such known compounds as cyclobenzaprine and dantrolene and their salts.

The preferred embodiment of this invention has four major components:
(1) Component 1 is the combination of NSAIDs, from 3-20%, which, for example, in one preferred form may be: Ibuprofen, 5% Diethylamine salicylate, 3% Indomethacin, 3% Diclolfenac, 1% Mefenamic acid, 2% Diflunisal, 2% Piroxicam, 3%.
(2) Component 2 is the chemical that enhances penetration of drugs across skin, which, for example, in preferred form may be: Dimethylsufoxide 3% or Sodium hyaluronate 2.5%.
(3) Component 3 is a chemical that enhances blood flow to the site of application, which, for example, in a preferred form may be: Capsaicin 0.025%.
(4) Component 4 is a smooth muscle relaxant, for example 1% cyclobenzaprine hydrochloride or 1% dantrolene sodium.
(5) Component 5 is the base that carries the product to the site of application. Following are examples of some of these preferred embodiments. However, anyone skilled in the art of formulation would soon realize the importance of selecting an appropriate base that will be compatible with other components, delivers drug at a desired rate, is pharmaceutically elegant and acceptable to patient. Examples include:
   Example 1: 95% ethanol 30% propylene glycol 10.0% Carbopol (4% aqueous solution) 940 25.0% triethanolamine 2.0%, Purified water qs to 100%
   Example 2: Carbomer 934P 2.0% Triethanolamine 1.3% Ethanol 17.1% Propylene Glycol 10.0% Ethoxydiglycol 2.5% C12-15 benzyl alcohols 1.2% Aloe vera gel. Purified Water qs to 100%.
   Example 3: Coconut fatty acid diethanolamide 2.0% Gelatin 3.0% D-sorbitol 30.0% Light anhydrous silicic acid 2.0% Oleyl alcohol 1.0% Propylene glycol 7.0% Sodium polyacrylate 5.0% Sodium carboxymethylcellulose 1.0% Polybutene 1.0% Tartaric acid 0.15% Aluminum hydroxide 0.2 Purified water qs to 100%
   Example 4: Coconut fatty acid diethanolamide 2.0% Oleyl alcohol 1.0% 1,3-Butylene glycol 10.0% D-sorbitol solution (70%) 30.0% Sodium polyacrylate 4.0% Sodium carboxymethylcellulose 1.0% Polybutene 1.0% Light anhydrous silicic acid 2.0% Tartaric acid 0.15% Aluminum glycinate 0.5% Purified water qs to 100%.
   Example 5: Coconut fatty acid diethanolamide 2.0% Oleyl alcohol 1.0% D-sorbitol 20.0% Hydroxypropylcellulose 2.0% Purified water qs to 100%
   Example 6: Coconut fatty acid diethanolamide 2.0% Polyethylene glycol 5.0% 1,3-Butylene glycol 5.0% Ethanol 5.0% Isopropanol 5.0% POE hydrogenated castor oil 5.0% POE lauryl ether 5.0 Pluronic F68 5.0% Purified water qs to 100%
   Example 7: Coconut fatty acid diethanolamide 4.00% Glycerin 5.00% Isopropyl alcohol 30.00% Hydroxyethylcellulose 2.00% Methylcellulose 2.00% Oleyl alcohol 1.00% Oxybenzone 0.50% Methylparaben 0.15% Propylparaben 0.50% Purified water qs to 100%.
   Example 8: Diethyl sebacate 3.0% Isopropyl alcohol 40.0% Hydroxypropyl cellulose 2.5% Tartaric acid q.s. to 100%
   Example 9: Diisopropyl adipate 4.0% Ethanol 40.0% Hydroxypropyl cellulose 2.5% Citric acid q.s. Purified water q.s. to 100%
   Example 10: Diisopropyl adipate 5.0% Lactic acid 0.04% isopropyl alcohol 40.0% Hydroxypropyl cellulose (H) 4.0% Hydroxypropyl cellulose 4.5% Purified water qs to 100%.
   Example 11: Glycerin 5% Benzyl Alcohol 3% QS with water to 100% Isopropyl alcohol, water carbopol-based cremagel (to give consistency of Voltaren Emulgel®)
   Example 12: Lauric acid diethanolamide 1.0% Coconut fatty acid diethanolamide 1.0% Polysorbate 60 3.0% Cetanol 4.0% Stearyl alcohol 5.0% Octyidodecanol 5.0% Medium-chain fatty acid triglyceride 6.0% Sorbitol 10.0% Butylhydroxyanisole 0.01% Methylparaben 0.1% Propylparaben 0.1% Purified water qs to 100%.
   Example 13: Lauric acid diethanolamide 2.0% Hydroxypropylcellulose 0.5% Pluronic F-68 1.0% Propylene glycol 2.0% Isostearyl alcohol 5.0% Oxybenzone 0.5% Methylparaben 0.15% Propylparaben 0.05% Purified water qs to 100%.
   Example 14: Lauric acid diethanolamide 4.0% Oleyl alcohol 1.0% Glycerin 5.0% Isopropyl alcohol 30.0% Hydroxyethylcellulose 2.0% Methylcellulose 2.0% Oxybenzone 0.5% Methylparaben 0.15% Propylparaben 0.05% Purified water qs to 100%
   Example 15: Lauric acid diethanolamide 5.0% Coconut fatty acid diethanolamide 5.0% Oleyl alcohol 3.0% Hydroxypropylcellulose 0.5% Pluronic F68 1.0% Propylene glycol 2.0% Oxybenzone 0.5.0% Methylparaben 0.15% Propylparaben 0.05% Purified water qs to 100%
   Example 16: Light anhydrous silicic acid 2.0% Sodium polyacrylate 4.0% Carmellose sodium 2.0% Hydroxypropylmethylcellulose 2.0% Coconut fatty acid diethanolamide 2.0% Oleyl alcohol 1.0% 1,3-Butylene glycol 10.0% 70% Sorbitol solution 30.0% Aluminum glycinate 0.2% Tartaric acid 0.3% Purified water qs to 100%
   Example 17: Phlojel® (Aqueous lecithin gel)
   Example 1: Phlojel® Ultra Base (Organic lecithin gel)
   Example 18: Purified oleic acid (Extraolein 90 2.0% Diisopropyl adipate 2.0% ODO (octyidecyl octyltriglyceride) 4.0% Glycerine 5.0% 1,3-Butylene glycol 7.0% POE (60 mole) hardened castor oil 1.5% Preservative q.s. Carboxyvinyl polymer 0.2% Potassium hydroxide 0.14% Purified water qs to 100%
   Example 19: Sodium polyacrylate 4.00% Carmellose sodium 2.00% Hydroxypropylmethylcellulose 2.00% Lauric acid diethanolamide 2.00% Lauryl alcohol 1.00% Propylene glycol 10.00% 70% Sorbitol solution 30.00% Aluminum hydroxide 0.20% Tartaric acid 0.30% Purified water qs to 100%

What is claimed is:

1. A pharmaceutical composition for use in the treatment of pain and inflammation and the treatment of muscle spasms and associated pain, soreness and tightness of muscles in a mammalian organism and adapted for topical administration, said composition comprising: (i) an analgesically and anti-inflammatory effective amount of at least one drug chosen from each of the groups of NSAIDs consisting of propionic acid derivatives, acetic acid derivatives, fenamic acid derivatives, biphenyl carbolic acid derivatives, oxicam derivatives and (ii) an amount effective in the treatment of muscle spasms, and associated symptoms, of a muscle relaxant selected from the group consisting of cyclobenzaprine, chlorzoxanzone, methocarbamol, dantrolene and the pharmaceutically acceptable salts thereof.

2. The composition of claim 1, where the composition further contains an enhancer of drug penetration selected from the group comprising of dimethylsufoxide, hyaluronic acid, their salts and homologues thereof, C4 to C16 aliphatic group substituted acetals, hemi-acetals and morpholines, alcohols, glycols, lactams, urea, cycloethylene urea, 1,3-dioxolone, 2-methyl-1-3-dioxolone, 1,3-dioxane, 2 methyl-1,3-dioxane, morpholine, N-methylmorpholine, N-dimethylformamide, methylacetate, ethyllactate, monosaccharides, polysaccharides, amino acids, amino alcohols, diethylamine, cycloethylene carbonate, 1-dodecylazacycloheptan-2-one hexamethylenelauramide, N-methyl-2-pyrrolidone, and sucrose aliphatic acid ester.

3. The composition of claim 1 wherein the composition further contains an enhancer of blood flow selecting from the group comprising of capsaicin, capsicum extract; erucic acid; nicotinic acid salts; nicotinic acid esters, nicotinyl alcohols, mustard oil, menthol, methyl salicylate, hydrallazine, verapamil, diltiazem, felodipine, minoxidil, amlodipine, glyceryl trinitrate, isosorbide mononitrate, nicorandil, dipyridamole, alprostadil, oxpentifylline, hydroxyethyl rutosides, tartrazine, adenosine and nimodipine.

4. The composition of claim 1 wherein the said propionic acid derivative is ibuprofen.

5. The composition of claim 1 wherein the said acetic acid derivative is diclofenac.

6. The composition of claim 1 wherein the said fenamic acid derivative is mefenamic acid.

7. The composition of claim 1 wherein the said biphenyl carbolic acid derivative is diflunisal.

8. The composition of claim 1 wherein the said oxicam derivative is piroxicam.

9. The composition of claim 1 wherein the said is an ointment, cream, poultice, dressing, gel, or devices for instant, slow or programmed released to the site of application.

10. The composition of claim 1 wherein the said muscle relaxant is cyclobenzaprine hydrochloride.

11. The composition of claim 1 wherein the said muscle relaxant is dantrolene sodium.

12. A pharmaceutical composition consisting essentially of a pharmaceutical base containing of (i) from 1-10% of each of ibuprofen, diclofenac, piroxicam, mefenamic acid, diflunisal and piroxicam, (ii) 1-10% dimethylsufoxide (iii) 0.1-0.5% capsaicin (iv) 0.1-10% cyclobenzaprine.
